(19) United States
(12) Patent Application Publication
(10) Pub. No.: US 2005/0148523 A1
(43) Pub. Date: Jul. 7, 2005

(54) Method of treating HIV infection in atazanavir-resistant patients using a combination of atazanavir and another protease inhibitor

(76) Inventors: Richard J. Colonno, Farmington, CT (US); Jacques Friborg JR., Hamden, CT (US); Ronald E. Rose, Madison, CT (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 11/011,226

(22) Filed: Dec. 13, 2004

Related U.S. Application Data
(60) Provisional application No. 60/532,746, filed on Dec. 23, 2003.
(60) Provisional application No. 60/529,678, filed on Dec. 15, 2003.

(51) Int. Cl. A61K 31/7056; A61K 31/551

(52) U.S. Cl. 514/43; 514/220

(57) ABSTRACT

A method of treating HIV infection in a human patient wherein the infecting HIV strain has become resistant to atazanavir, the method comprising administration of a therapeutically effective amount of a combination of atazanavir or a pharmaceutically acceptable salt thereof, and at least one other HIV protease inhibitor. A method for enhancing the effectiveness of a second HIV protease inhibitor in treating HIV infection in a human patient whose HIV strain has become resistant to atazanavir or a pharmaceutically acceptable salt thereof, comprising administering to said human patient an amount of atazanavir or a pharmaceutically acceptable salt thereof effective in maintaining the resistant strain, in combination with the second HIV protease inhibitor. The resistance to atazanavir in the human is manifested by the existence of the signature mutation consisting of I50L mutation in the HIV protease.

### CROSS-REFERENCE TO RELATED APPLICATIONS

This application claims the benefit of U.S. Provisional Application Ser. No. 60/532,746 filed Dec. 23, 2003 and 60/529,678 filed Dec. 15, 2003.

### BACKGROUND OF THE INVENTION

1. Field of the Invention

A method of treating HIV infection in a human patient wherein the infecting HIV strain has become resistant to atazanavir, the method comprising administration of a therapeutically effective amount of atazanavir or a pharmaceutically acceptable salt thereof, and a therapeutically effective amount of at least one other HIV protease inhibitor.

A method for enhancing the effectiveness of a second HIV protease inhibitor in treating HIV infection in a human patient whose HIV strain has become resistant to atazanavir or a pharmaceutically acceptable salt thereof, comprising administering to said human patient an amount of atazanavir or a pharmaceutically acceptable salt thereof effective in maintaining the resistant strain, in combination with a therapeutically effective amount of the second HIV protease inhibitor.

The resistance to atazanavir in the human is manifested by the existence of the signature mutation consisting of I50L mutation in the HIV protease.

2. Background Art

HIV-1 (human immunodeficiency virus-1) infection remains a major medical problem, with an estimated 42 million people infected worldwide at the end of 2002. The number of cases of HIV and AIDS (acquired immunodeficiency syndrome) has risen rapidly. In 2002, ˜5.0 million new infections were reported, and 3.1 million people died from AIDS. Currently available drugs for the treatment of HIV include nine nucleoside reverse transcriptase (RT) inhibitors or approved single pill combinations (zidovudine or AZT (or Retrovir®), didanosine (or ddI or Videx®), stavudine (or d₄T or Zerit®), lamivudine (or 3TC or Epivir®), zalcitabine (or DDC or Hivid®), abacavir succinate (or Ziagen®), Tenofovir disoproxil fumarate salt (or Viread®), Combivir® (contains-3TC plus AZT), Trizivir® (contains abacavir, lamivudine, and zidovudine); three non-nucleoside reverse transcriptase inhibitors (NNRTI): nevirapine (or Viramune®), delavirdine (or Rescriptor®) and efavirenz (or Sustiva®), and eight peptidomimetic protease inhibitors or approved formulations: saquinavir, indinavir, ritonavir, nelfinavir, fosamprenavir, amprenavir, lopinavir, Kaletra®(lopinavir and ritonavir), and atazanavir (Reyataz®). Each of these drugs can only transiently restrain viral replication if used alone. However, when used in combination, these drugs have a profound effect on viremia and disease progression. In fact, significant reductions in death rates among AIDS patients have been recently documented as a consequence of the widespread application of combination therapy. However, despite these impressive results, 30 to 50% of patients ultimately fail combination drug therapies. Insufficient drug potency, non-compliance, restricted tissue penetration and drug-specific limitations within certain cell types (e.g. most nucleoside analogs cannot be phosphorylated in resting cells) may account for the incomplete suppression of sensitive viruses. Furthermore, the high replication rate and rapid turnover of HIV-1 combined with the frequent incorporation of mutations, leads to the appearance of drug-resistant variants and treatment failures when sub-optimal drug concentrations are present.

Therefore, novel anti-HIV agents and methods to overcome drug resistance in the infecting HIV strain or enhance the effectiveness of other HIV treating drugs are needed to provide more treatment options.

Y. F. Gong et al, Antimicrob. Agents and Chemotherapy, Vol. 44, No. 9, pp. 2319-2326 (September 2000) has suggested that BMS-232632 (i.e. atazanavir) may be a valuable protease inhibitor for use in treating HIV infections, as well as observing the I50L substitution mutation caused by atazanavir in the HIV protease [10].

Several prior art publications have subsequently established the unique ISOL mutation in the HIV protease as the signature resistance mutation when patient derived isolates become resistant during treatment with atazanavir containing regimens (see R. Colonno et al, presentation made Jul. 2-6, 2002, Seville, Spain, at 6^{th} International Conference Workshop on HIV Drug Resistance entitled “Identification of Amino Acid Substitutions Correlated with Reduced Atazanavir Susceptibility in Patients Treated with Atazanavir Containing Regimens” [33].

### SUMMARY OF THE INVENTION

It has now been surprisingly and unexpectedly found that when patients have become resistant to further treatment with the HIV protease inhibitor, atazanavir, because of virologic failure (i.e. the HIV viral load in the human is no longer kept below acceptable limits by further treatment with atazanavir), continuing treatment with another HIV protease inhibitor in combination with atazanavir is a useful treatment for HIV infection.

Specifically, the invention includes a method of treating HIV infection in a human patient wherein the infecting HIV strain has become resistant to atazanavir, the method comprising administration of a therapeutically effective amount of atazanavir or a pharmaceutically acceptable salt thereof, and a therapeutically effective amount of at least one other HIV protease inhibitor.

Another embodiment includes the method wherein said resistance is manifested by the existence of a signature mutation consisting of the I50L mutation in the HIV protease and the amount of atazanavir or a pharmaceutically acceptable salt thereof is sufficient to maintain the existence of the I50L mutation in the HIV protease.

Another embodiment includes the method wherein the other HIV protease inhibitor is selected from the group consisting of saquinavir, ritonavir, indinavir, nelfinavir, amprenavir, fosamprenavir and lopinavir.

Another embodiment includes the method wherein the amount of the other HIV protease inhibitor administered to the human patient in combination with the atazanavir or pharmaceutically acceptable salt thereof is less than the amount required in the absence of atazanavir.

Another embodiment includes the method wherein the other HIV protease inhibitor is selected from the group consisting of saquinavir, ritonavir, indinavir, nelfinavir, fosamprenavir, amprenavir and lopinavir.

Another embodiment includes the method for enhancing the effectiveness of a second HIV protease inhibitor in treating HIV infection in a human patient whose HIV strain has become resistant to atazanavir or a pharmaceutically acceptable salt thereof, comprising administering to said human patient an amount of atazanavir or a pharmaceutically acceptable salt thereof effective in maintaining the resistant strain, in combination with a therapeutically effective amount of the second HIV protease inhibitor.

Another embodiment includes the method wherein said resistance is manifested by the existence of a signature mutation consisting of the I50L mutation in the HIV protease and the amount of atazanavir or a pharmaceutically acceptable salt thereof is sufficient to maintain the existence of the I50L mutation in the HIV protease.

Another embodiment includes the method wherein said HIV protease inhibitor is selected from the group consisting of saquinavir, ritonavir, indinavir, nelfinavir, fosamprenavir, amprenavir and lopinavir.

Another embodiment includes the method wherein the amount of the other HIV protease inhibitor administered to the human patient in combination with the atazanavir or pharmaceutically acceptable salt thereof is less than the amount required in the absence of atazanavir.

Another embodiment includes the method wherein the other HIV protease inhibitor is selected from the group consisting of saquinavir, ritonavir, indinavir, nelfinavir, fosamprenavir, amprenavir and lopinavir.

### BRIEF DESCRIPTION OF THE FIGURES

FIG. 1. Phenotypic profiles of clinical isolates. Phenotype (expressed as FC) of baseline (open circles) and on-treatment (closed circles) clinical isolates from patients who developed an I50L substitution are plotted for each of the seven PIs. Samples with mixtures of Ile and Leu at position 50 are indicated as gray circles. An FC=1 is reflective of wild type susceptibility.

FIG. 2. Susceptibility Profiles of Recombinant Viruses Containing an I50L Substitution. Phenotype (open circles) of all NL4-3 recombinant viruses listed in Table 2 were determined (N≧4 assays) for each PI and expressed as fold difference from the reference NIA-3 virus. An FC=1 is reflective of wild type susceptibility.

FIG. 3. Susceptibilities of Clinical Isolates Containing I50L or D30N Substitutions. Median susceptibility (FC) of clinical isolates containing either an I50L (n=16, white bars) or D30N (n=75, gray bars) in the absence of other protease resistant mutations. An FC=1 is reflective of wild type susceptibility.

FIG. 4. Growth Curves of Recombinant Viruses Containing I50L Substitutions. Replication fitness of RF and NL4-3 recombinant viruses containing a wild-type protease gene (circle), or the variants I50L (diamond), A71V (square) and I50L/A71V (triangle) were determined in parallel infections by monitoring the levels of p24 antigen production over time in the supernatants. Results are from 2 independent experiments following infection of cells at an MOI of 0.001 with NL4-3 virus (panel A) and RF virus (panel B) or at an MOI of 0.005 with NL4-3 virus (panel C) and RF virus (panel D).

### DETAILED DESCRIPTION OF INVENTION

HIV protease inhibitors (PIs) are potent and effective antiretrovirals. However, their extensive use has led to the emergence of HIV-1 variants exhibiting cross-resistance to multiple PIs [1, 2, 11]. The correlation between genotypic changes within the protease (PR) gene and phenotypic resistance remains poorly understood and secondary substitutions appear to play a major role in expression of a resistance phenotype [3-5 and 11].

Atazanavir (ATV, Reyataz®, BMS-232632) is a once daily HIV-1 PI [6-9] that was recently approved by the U.S. FDA for combination therapy in HIV-1 infected patients. ATV has a 50% effective concentration (EC₅₀) of 3 to 5 nM against a variety of HIV-1 isolates in different cell types and is a highly selective and effective inhibitor of the HIV-1 protease in vitro (Ki of <1 nM) [7]. In vitro passage of HIV-1 in the presence of ATV results in the selection of resistant variants [10]. Genotypic analysis of three different HIV-1 strains with in vitro-selected resistance to ATV indicated that a N88S substitution in the viral protease appeared first during the selection process in two of the three strains. An I84V change appeared to be an important substitution in the third strain, along with I50L, and all three variants required multiple changes to achieve significant resistance (>10-fold decrease in susceptibility) levels. Mutations were also observed at the protease cleavage sites following drug selection. The evolution to resistance was somewhat distinct for each of the three strains utilized, suggesting that multiple pathways to resistance are possible and confirming the importance of viral genetic background in resistance development [10].

The susceptibility profile of ATV was subsequently determined using a panel of 950 clinical isolates exhibiting a wide array of PI resistance profiles and genotypic patterns [11]. In general, reductions in ATV susceptibility required several amino acid changes, were modest in degree, and susceptibility was retained among isolates resistant to one or two other PIs. While ATV displayed a distinct resistance pattern relative to the six PIs tested, there was a clear trend toward loss of susceptibility to ATV as isolates exhibited increasing levels of cross-resistance to multiple PIs with the percentage of isolates resistant to 1, 2, 3, 4 or 5 PIs remaining susceptible to ATV determined to be 88%, 81%, 34%, 16% and 5%, respectively. Genotypic analysis of 943 PI susceptible and resistant clinical isolates identified a correlation between the presence of specific amino acid substitutions in the viral protease (L10I/V/F, K20R/M/I, L24I, L33I/F/V, M36I/L/V, M46I/L, G48V, I54V/L, L63P, A71V/T/I, G73C/S/T/A, V82A/F/S/T, I84V and L90M) and decreased susceptibility to ATV. While no single substitution or combination of substitutions appears to be predictive of ATV resistance, the presence of at least 5 of these substitutions correlated strongly with decreased ATV susceptibility.

The primary objective of the current study was to characterize the phenotype profile and emerging genotype(s) of clinical isolates from PI-naïve patients who were designated as virologic failures and developed resistance to ATV while receiving ATV-containing regimens.

### Materials and Methods

Compounds. ATV was synthesized at Bristol-Myers Squibb. ATV can be made according to the procedures disclosed in U.S. Pat. No. 5,849,911 which is incorporated by reference in its entirety. All of the other drugs utilized in this study were either purchased and or provided by the central testing laboratories and purified to the following active form: free base-atazanavir, amprenavir, indinavir, nelfinavir and saquinavir; neutral compound-lopinavir and ritonavir.

Study Population. Viral samples (baseline and on-treatment, where available) were obtained from patients who experienced virologic failure while enrolled in studies AI424-007/041 [12] (ATV+d4T and ddI), AI424-008/044 [13, 14] (ATV+d4T and 3TC) and AI424-034 [9] (ATV or EFV in combination with ZDV and 3TC). Subjects designated as virologic failures included subjects experiencing a viral rebound (generally defined as a confirmed increase in HIV RNA to levels above 400 c/mL) and non-responders (did not achieve HIV RNA values below 400 c/mL by week 16 or 24 (study AI424-034)) had paired plasma specimens from baseline and on-treatment evaluated for phenotypic and genotypic changes. In general, virologic failure specimens were selected for further resistance evaluation only if they contained >1,000 copies/mL HIV-1 RNA and there was no indication that the specimen came from a patient at a time of transient non-adherence to study drug(s). On-treatment samples from patients experiencing a confirmed viral rebound were collected from the first visit demonstrating the rebound; if the RNA level of that sample was <1000 c/mL, the next available sample with RNA >1000 c/mL was used. For non-responders, samples from week 16 (or 24) were tested. Drug adherence was determined from pill counts of returned study medications at each scheduled visit.

Cells and Viruses. MT-2 cells, HIV-1 RF, HIV1-LAI, and the HIV-1 NL4-3 proviral clones were obtained through the AIDS Research and Reference Reagent Program, National Institutes of Health. MT-2 cells were cultured in RPMI 1640 medium supplemented with 10% fetal bovine serum, 2 mM L-glutamine, and 10 mM Hepes buffer. HIV-1 viruses were generated by transient transfection of the proviral DNA into Human Embryonic Kidney 293 cells using Lipofectamine reagents (Gibco BRL). The viruses were harvested 72 hr post-transfection, amplified in MT-2 cells, and titered in the same cells using a virus yield assay [15].

Recombinant HIV-1 Variants and Chimeric NL4-3 Proviral Clones. HIV-1 strains with defined substitutions in the protease gene were generated by QuikChange® site-directed mutagenesis as recommended by the manufacturer (Stratagene). Recombinant NL4-3 and LAI proviral clones containing the wild-type and mutant protease genes from RF and clinical isolates were constructed by replacing the SseI/ApaI fragment of pNL4-3 with the appropriate SseI/ApaI protease fragments.

Drug Susceptibility Assays. Clinical isolates were subjected to phenotypic (PhenoSense™) and genotypic (GeneSeq™) analysis by Virologic, Inc. as described elsewhere [16, 17]. Over the time period (several months) in which the samples reported here were tested, the ATV IC₅₀ measured for the NL4-3 reference strain ranged from 0.9-2.0 nM. The success rate in obtaining phenotype and genotype results from patients experiencing virologic failure ranged from 30 to 50%, largely because of low HIV RNA levels (the assays used required levels of >=500 c/mL), insufficient sample volume, or no sample was available.

Recombinant viruses (generated separately from those made for PhenoSense assays) were tested in an MT-2 cell susceptibility assay at Bristol Myers Squibb. MT-2 cells were infected with recombinant viruses at a multiplicity of infection (MOI) of 0.005 50% tissue culture infective doses (TCID₅₀)/cell followed by incubation in the presence or absence of serially diluted protease inhibitors for 4 to 7 days. Virus yields were quantified using a RT assay as previously described [18]. The results from at least four independent experiments were used to calculate the EC₅₀.

Growth Properties of Mutant Viruses. The replication kinetics and fitness of recombinant mutant viruses were evaluated in parallel following the infection of MT-2 cells at an MOI of either 0.001 or 0.005 TCID₅₀/cell. Briefly, following viral inoculation for 1 hr, the cells were washed with RPMI and further cultured in fresh RPMI complete media for up to 15 days post-inoculation in the absence of inhibitors. Supernatant fractions were harvested every day and frozen at −80° C. until assayed. Virus replication was determined by measuring the level of p24 (ELISA) in the supernatant fractions.

### Results

Identification of I50L as the Signature Mutation for ATV Resistance. Previous in vitro studies indicated that ATV has an overall resistance profile distinct from other PIs [10, 11]. To better understand how viruses become resistant in ATV-treated patients, clinical isolates from treatment naïve patients experiencing virologic failure while enrolled in clinical studies AI424-007/041, AI424-008/044, and AI424-034 were analyzed. Table 1 provides a summary of the outcome metrics from these clinical studies. Overall, the detection of ATV resistant variants appeared to be low (˜2% of treated patients) and was comparable to, if not somewhat lower than, that of the study control drugs nelfinavir (NFV) and efavirenz (EFV). ATV resistance was defined as ≧2-fold decrease in ATV susceptibility from baseline, a Fold Change (FC) of ≧2.5 (relative to the Nl4-3 reference strain), or evidence of an isoleucine (Ile) to leucine (Leu) change at amino acid residue 50 (I50L). Resistance levels for NFV and EFV were also defined as a FC reaching 2.5.

Phenotypic analysis of virus samples from the 208 patients designated as virologic failures on ATV containing regimens initially identified 14 pairs of isolates whose on treatment virus exhibited a >2-fold decrease in ATV susceptibility relative to baseline. Genotypic analysis of these isolates showed that all on-treatment isolates contained an I50L substitution in the absence of other recognized primary mutations within the viral protease. Nine also showed emergence of an A71V substitution. The I50L substitution has not previously been reported in patients, while A71V is a common secondary PI mutation and naturally occurring polymorphism. An additional four on-treatment isolates from patients designated as virologic failures also exhibited an ATV-specific resistance phenotype and had the I50L change, however baseline isolates were unavailable. Three additional isolates with an I50I/L mixture at residue 50 and susceptible phenotypes, as well as three other on-treatment I50L-containing isolates with no available phenotype data were reported from patients not designated as virologic failures. Thus, there are 24 patient isolates with either genotypic and/or phenotypic data implicating an I50L substitution as a primary substitution in ATV resistance.

Amino acid substitutions in the P7-P1 and P1-P6 processing sites of the gag precursor have been detected in vitro and in vivo during treatment with HIV-1 PIs [10, 19-25]. To determine whether similar mutations at the P7-P1 and P1-P6 cleavage sites developed during treatment with ATV, we examined the gag sequence that spans the P7-P1-P6 junctions of the seven available ATV-resistant viruses from trial AI424-007. The substitution A431V at the P2 position of the P7-P1 cleavage site associated with IDV and RTV resistance [21-23] emerged on treatment in three viruses, while changes were not observed at these cleavage sites in the remaining four viruses. These results suggest that cleavage site changes are not required for the expression of the ATV resistance phenotype.

Appearance of I50L was noted as early as treatment week 16, although the mean and median time on study to emergence of resistance were 62 and 53 weeks, respectively, and the mean time to virologic failure in this group of patients (N=10) was 50 weeks. ATV phenotypic resistance levels ranged from 1.5 to 29 relative to the NL4-3 reference strain, with a median FC of 9.6-fold, with increased susceptibility to the other PIs tested consistently noted. Lamivudine resistance related to emergence of a M184I/V substitution was observed in all 11 I50L isolates from patients designated as virologic failures whose treatment regimen contained lamivudine.

Emergence of I50L occurred in a variety of genetic backgrounds, with baseline isolates averaging nearly seven protease substitutions, including L63P, M36L, L10V and K20R. The I50L substitution was frequently accompanied by an A71V change (56%) and to a lesser degree by K45R (33%), I64V (28%), G73S (22%) and M46I(17%). The strong relationship between emergence of I50L and ATV resistance development was further supported by two isolates in which the progression of an I50 to I50I/L to I50L correlated directly with increasing levels of the ATV resistance (data not shown). Where viral subtype information was available, I50L containing isolates were observed among both subtype B (n=17) and C (n=6) viruses.

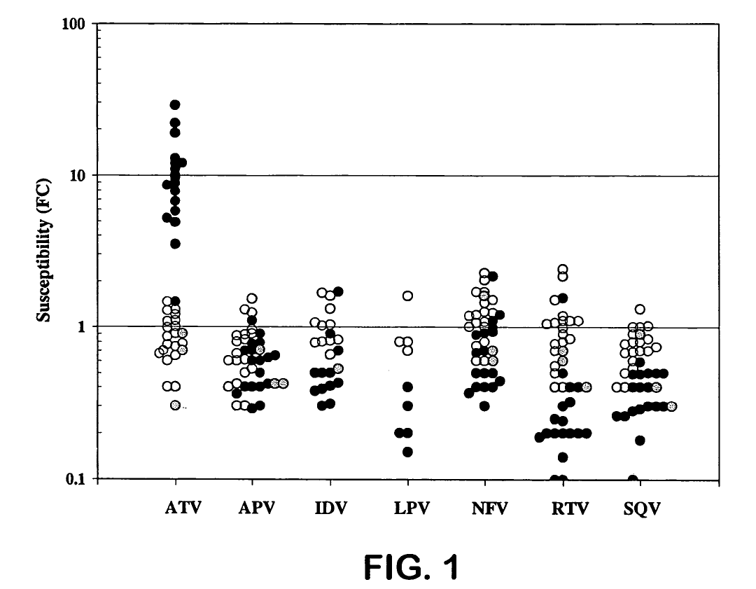

Effect of I50L on Susceptibility to ATV and Other PIs. Emergence of I50L correlated with ATV specific resistance, while the susceptibility to other PIs was either unchanged or increased. The median decrease in ATV susceptibility from baseline was 9.6-fold (n=15), whereas, the median susceptibility from baseline increased 1.5-fold (n=15) for APV, 2.0-fold (n=9) for IDV, 3.2-fold (n=4) for LPV, 1.6-fold (n=15) for NFV, 4.1-fold (n=15) for RTV and 2.0-fold (n=15) for SQV. Susceptibilities to APV and NFV displayed more modest increases in susceptibility compared to the other PIs, although APV FC values were mostly below 1 at baseline. Susceptibility (FC) levels, relative to the reference strain, reached 0.4-fold or lower in 33-100% of the I50L isolates: APV (39%), IDV (58%), LPV (100%), NFV (33%), RTV (83%) and SQV (72%). In contrast, the percentage of baseline isolates exhibiting a FC≦0.4 ranged from 21% for APV to either 10% (RTV, SQV) or 0% (IDV, LPV, NFV). This overall phenotypic profile of selective resistance to ATV and increased susceptibility to the other PIs is clearly evident in FIG. 1, where the baseline (open circles) and on-treatment (black circles) phenotypes of isolates is displayed. Three on treatment isolates exhibiting ATV FC values <1 contain I50I/L mixtures gray circles. The one isolate displaying a FC of only 1.5 exhibited a FC of 0.2 prior to developing the I50L change.

ATV-Specific Resistance Maps Exclusively to the I50L Substitution. Direct evidence for the role of the I50L substitution in resistance to ATV was obtained using a series of clonal NL4-3 recombinant viruses containing only the protease gene from several resistant clinical isolates originating in study AI424-007. The ATV genotypes and phenotypes in MT-2 cells of this panel of recombinant viruses are shown in Table 2. These results demonstrate that the ATV resistant phenotype is carried within the coding sequence of the viral protease and does not require either cleavage site changes or modifications in other viral proteins. To further demonstrate the specific impact of the I50L substitution on ATV resistance, the I50L and A71V substitutions alone and in combination were inserted into the wild-type protease gene of three recombinant viral strains. Decreases in ATV susceptibility of 2.1 to 5.4-fold were observed in all three viral backbones containing only the I50L substitution, and 5.7 to 10-fold with the combination of I50L and A71V (Table 2). In contrast, presence of the A71V alone resulted in ATV susceptibility changes of 0.9 to 2.4-fold. These results provide the most convincing evidence that the I50L alone is critical to expression of the observed ATV resistance phenotype and that the degree of resistance is enhanced by secondary substitutions such as A71V. Overall, the results strongly suggest that the I50L substitution, sometimes combined with an A71V or other changes, is the signature mutation for ATV resistance.

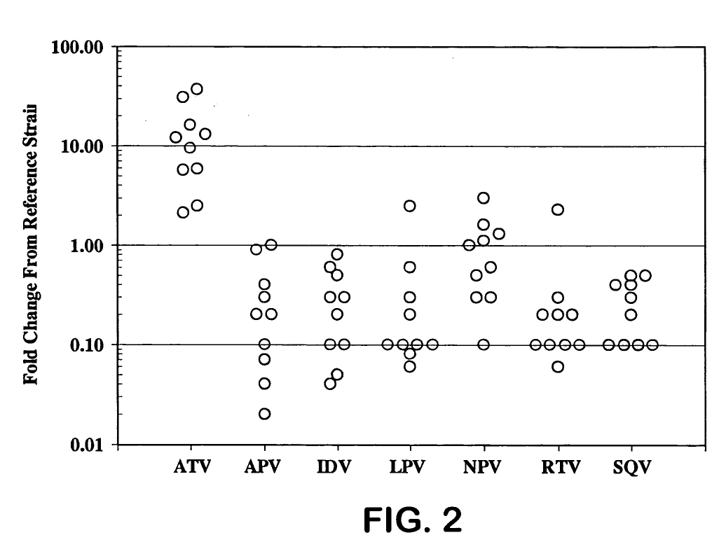

Phenotypic profiling of these recombinant viruses was performed to determine if the I50L induced resistance to ATV was also responsible for the enhanced susceptibility to other PIs. FIG. 2 displays the change in susceptibility to all PIs compared to the NL4-3 reference strain for each of NL4-3 recombinant viruses listed in Table 2. Results closely resemble what was previously observed with clinical isolates (FIG. 1), ATV specific resistance and a strong tendency toward increased susceptibility to the other PIs. Recombinant clones containing the baseline protease genes for these resistant patient isolates showed little change from the NL4-3 reference strain. Moreover, enhanced susceptibility to APV, IDV, LPV, NFV, RTV and SQV was observed in all three viral strains when the I50L or I50L/A71V substitutions were introduced by site-directed mutagenesis. In many cases, the increased susceptibilities of these recombinant viruses were ≧10-fold of the reference viruses.

Stability of Viruses Containing the I50L Substitution. Genotypic and phenotypic progression following the emergence of an I50L substitution was monitored in a small series of isolates derived from 4 patients who continued on an ATV containing regimen. Results (Table 3) show that continued therapy with ATV did not result in a rapid progression to broad cross-resistance or accumulation of additional amino acid changes despite continued ATV treatment for 12 to 52 weeks. Apart from patient 100, who continued ATV therapy for an additional year, the observed genotypic changes appeared to be relatively benign and consistent with the susceptibility phenotypes that were available. The newly emerging K45R, V82A and I93L substitutions observed in patient 100 were potentially of a greater concern because the V82A change is usually associated with cross-resistance to multiple PIs. However, all four patients showed stable viral loads ranging between 1,350 and 6,659 copies.

No Evidence of Cross-Resistance between ATV and APV. Because an I50V substitution is a signature mutation sometimes associated with APV resistance [26], the cross-resistance relationship between ATV and APV was further examined. As noted above, none of the I50L containing clinical isolates showed evidence of APV resistance (FIG. 1). A reciprocal evaluation was performed on a small panel of 8 clinical isolates that contained an I50V and were resistant (4.2 to 14-fold) to APV and either LPV or RTV, since isolates displaying APV specific resistance were not available. Seven of the eight isolates resistant to APV had an ATV FC less than 1.0, and 3 had an ATV FC of 0.4 or less (Table 4). Thus, a specific change at residue 50, to either Leu or Val, in the absence of other key primary and secondary substitutions, results in specific resistance to ATV and APV, respectively.

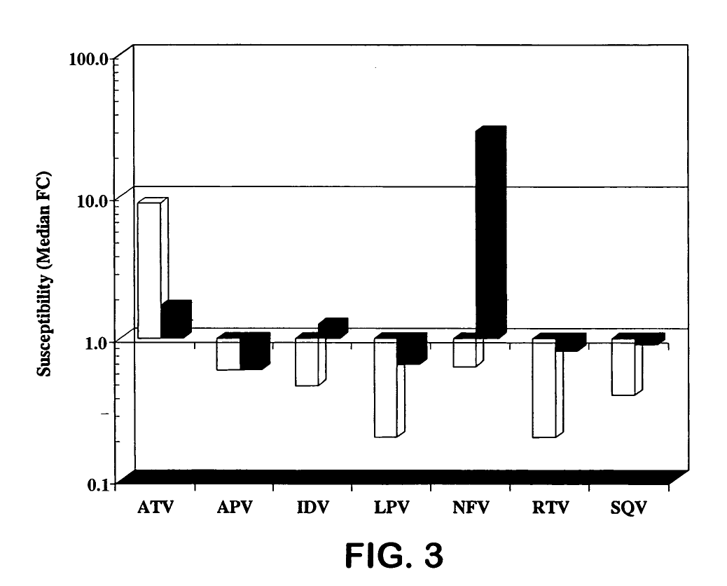

Comparative Studies of I50L and D30N Containing Viruses. The D30N substitution is a key signature mutation for NFV and reported to have little effect on susceptibility to other PIs. An important distinction between the I50L and D30N substitutions is that the I50L change, to date, has been observed 100% of the time when ATV phenotypic resistance was evident in the treatment-naive patients. In contrast, the D30N resistance pathway accounts for NFV resistance about 50% of the time [27]. A similar outcome was also observed in clinical studies AI424-007 and AI424-008 in which NFV was used as the PI control (Table 1). Comparative studies were performed on the 18 phenotyped viruses containing an I50L and 75 clinical isolates containing a D30N substitution without evidence of cross-resistance (FC≧2.5) to other PIs. Genotypic backgrounds for these 2 sets of viruses were very similar, with a wide variety of additional substitutions present (data not shown). The only statistically significant changes (p<0.001) based on Fisher's Exact Test were at residues 13, 30 and 88 for NFV and 50, 73 and 89 for ATV. The median FC value change from wt virus for each of these isolates vs. the seven approved PIs is presented in FIG. 3. While the I50L isolates displayed selective resistance to ATV and increased susceptibility to all of the other six PIs, the D30N containing isolates showed resistance to NFV and small median decreases in susceptibility to both ATV and IDV. The median FC of the D30N viruses was never below 0.4 for any of the other PIs, with only the APV susceptibility level reaching a FC as low as 0.6. In contrast, viruses containing the I50L substitution had median FC of 0.4 or less for IDV, LPV, RTV and SQV. These results suggest that the emergence of either an I50L or D30N substitution in this group of viruses will likely preserve future PI treatment options, but that the I50L change has a greater potential to increase susceptibilities to other PIs.

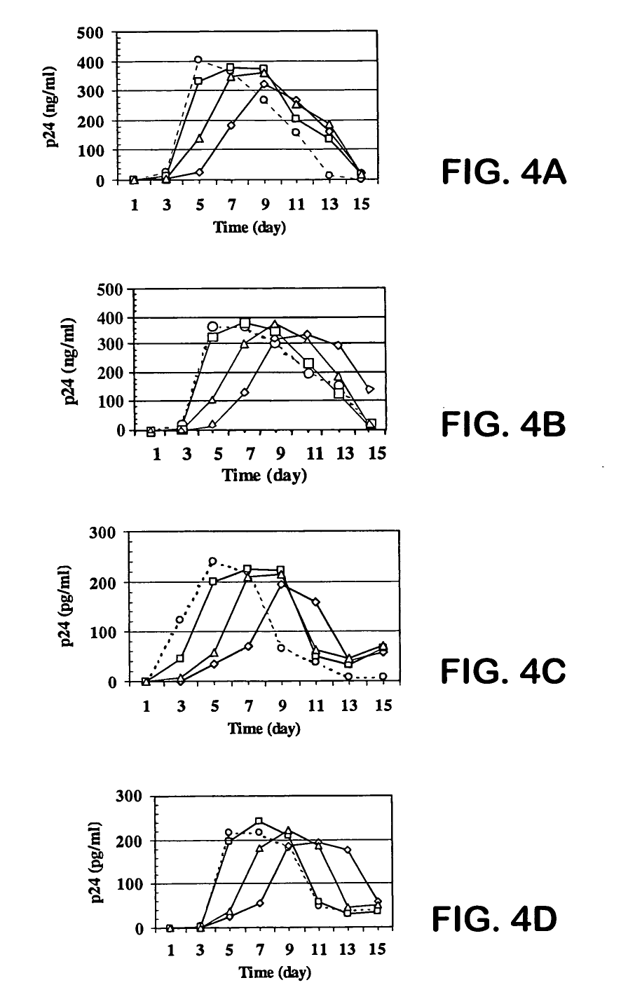

I50L Containing Viruses are Growth Impaired. Significant differences in HIV replication kinetics can be observed in parallel infections by measuring the amount of virus production over time via the detection of p24 antigen. To determine if the I50L substitution has an impact on viral replication, growth curves for the NL4-3 and RF recombinant viruses described in Table 2 were compared to their respective wild-type viruses. In each case (FIG. 4), insertion of the I50L substitution in either protease backbone resulted in a significant delay in viral replication, while addition of the A71V change with I50L restored some viability (FIGS. 4A and 4B). Insertion of the A71V substitution alone, however, had no significant effect on the rates of viral growth. The single-cycle replicative capacity (RC, [28]) of several I50L containing clinical isolates were also determined and ranged from 0.3 to 42% of the reference strain and in the three cases where paired baseline and on treatment isolates were available, the RC shifted from 92% to 4.2%, 150% to 12% and 85% to 37%. Together, these data suggest that the emergence of the I50L substitution impairs viral growth, most likely through a reduction in the enzymatic activity of the viral protease. The A71V change appears to be a compensatory substitution in this regard.

### Discussion

ATV is a potent inhibitor of HIV-1 protease with demonstrated effectiveness in clinical trials comparable to standard of care regimens such as efavirenz or nelfinavir [8, 9]. Its excellent oral bioavailability and pharmacokinetic profile enable once daily dosing and a low pill burden, in the absence of added RTV. Previous studies involving a large panel of viruses resistant to other PIs demonstrated that ATV has a distinct resistance profile relative to the other available PIs [11]. The current analysis was undertaken to identify the resistance markers and patterns of resistance in previously treatment-naïve patients treated with ATV containing regimens. Overall, viral variants resistant to ATV emerged infrequently in these patients. Despite the limited number of ATV resistant isolates available, an obvious correlation became apparent between the development of ATV phenotypic resistance in patients designated as virologic failures on ATV containing regimens and the emergence of a novel I50L substitution.

The I50L substitution is the signature resistance mutation for ATV. The appearance of I50L substitution is frequently accompanied by A71V, a secondary substitution also observed among isolates resistant to other PIs. Apart from a potential relationship with the K45R and G73S substitutions, none of the other observed amino acid changes appeared to correlate with ATV resistance. Supporting this conclusion is the observation that an ATV-resistant phenotype is expressed in all recombinant viral clones containing the I50L substitution in a variety of genetic backgrounds and independent of the A71V, K45R or G73S substitutions. This finding that recombinant viruses carrying only the I50L substitution express ATV-specific resistance also demonstrates that the cleavage site changes observed in vitro and in patient isolates are not required for expression of this resistance phenotype. However, the magnitude of resistance remains relatively low in the absence of other protease substitutions and an I50L substitution has not been observed in clinical isolates in the absence of other substitutions.

Amino acid residue 50 is located in the flap region of the HIV protease and plays a key role in enzymatic function and PI binding [29]. Seemingly small modifications, such as a change from the normal Ile to Leu or Val can result in ATV or APV resistance, and in the case of Leu, appears to significantly enhance susceptibilities to the other PIs.

The finding of an exclusive pathway to resistance among viral isolates from previously treatment-naive patients developing phenotypic resistance to ATV was somewhat surprising, since earlier in vitro selection studies [10] predicted the possibility of multiple pathways and that an N88S substitution would be a primary resistance marker. Those studies did, however, identify an I50L resistance pathway and demonstrated that viruses containing the I50L change were particularly susceptible to other PIs [10]. The increases in susceptibility are particularly noteworthy because they resulted in a high percentage of isolates having PI susceptibilities, as measured by FC, of ≦0.4. Clearly, selection of the I50L substitution is the most efficient in vivo pathway to escape the inhibitory pressure of ATV. Despite the decreased viral fitness resulting from selection of an I50L change, initial results suggest that viruses containing an I50L do not appear to rapidly accumulate additional amino acid changes or become cross resistant to multiple PIs (Table 3). Interestingly, the I50L substitution has also been observed (at a reduced frequency) in treatment-experienced patients in patients treated with ATV containing regimens.

The frequency and magnitude of the observed I50L changes are distinct from those observed for I50V and D30N, the signature resistance mutations for APV and NFV, respectively [27, 30, 31]. Viruses containing I50V or I84V substitutions displayed the greatest reductions in APV susceptibility. Four distinct genetic pathways to APV resistance have been described among clinical isolates from patients treated with APV: I50V, I54L/M, I84V and V32I+I47V [30]. Apart from the I84V pathway, the other three APV resistance pathways occur with near equal frequency and the most frequent substitution observed in the presence of I50V was M46I/L. Although minimal cross-resistance to other PIs is observed, I50V contributes to reduced susceptibility to RTV and LPV (lopinavir), and is associated with increased susceptibility to SQV and IDV in vitro [31]. In addition to I50V, selection of I54L/M appears to be somewhat unique to APV. In regard to development of resistance to NFV, a D30N substitution emerges in ˜50% of clinical isolates from patients on NFV (nelfinavir) regimens for a median of 13 weeks with a trend toward accumulation of additional amino acid changes at residues 35, 36, 46, 71, 77 and 88 [27]. The N88S substitution emerges in 20% of treated patients and can result in increased susceptibility to APV, although this effect can be neutralized by the presence of other substitutions such as M46I/L, L63P and V77I [32]. Isolates containing the D30N substitution as the only primary PI mutation remained susceptible to APV (amprenavir), IDV (idinavir), RTV (ritonavir) and SQV (saquinavir) [27].

It appears that treating HIV infection with protease inhibitors has been extended with the emergence of the I50L substitution resulting from prior treatment using ATV (atazanavir).

TABLE 1
Virological Outcomes and Resistance

| | Study | | | | | |
|---|---|---|---|---|---|---|
| Treatment arm (PI or | 007/041 | | 008/044 | | 034 | |
| NNRTI) | ATV | NFV | ATV | NFV | ATV | EFV |
| Patients Treated | 238 | 80 | 373 | 91 | 404 | 401 |
| Virologic Failures* (%) | 51 (21) | 18 (23) | 88 (24) | 27 (30) | 69 (17) | 69 (17) |
| Phenotyped/Genotyped | 25 | 9 | 27 | 8 | 26 | 20 |
| Resistance** (% of VF) | 12 (24) | 5 (28) | 4 (5) | 4 (15) | 8 (12) | 15 (22) |
| I50L (%) | 12 (100) | 0 | 4 (100) | 0 | 8 (100) | 0 |
| D30N (%) | 0 | 2 (40) | 0 | 3 (75) | 0 | 0 |
| K103N (%) | 0 | 0 | 0 | 0 | 0 | 11 (85) |

*Definition of failure described in Materials and Methods section.
**ATV resistance was defined as ≧2-fold decrease in ATV susceptibility from baseline, a Fold Change (FC) of ≧2.5 (relative to a reference strain), or evidence of an I50L amino acid change. Resistance levels for NFV and EFV were also defined as a FC reaching 2.5.

TABLE 2
Genotype and ATV Phenotype of Recombinant Viruses

| | | ATV |
|---|---|---|
| Virus | Amino Acid Substitutions | (Fold Change) |
| NL4-3 Control | | 0.8 ± 0.3 |
| NL4-3 398 | I50L + E35D, M36L, N37S, R41K, | 9.0 ± 1.5 (12) |
| | R57K, I62V, L63P, I93L | |
| NL4-3 347 | I50L + I13V, E35D, M36I, M46L, | 12.1 ± 1.4 (16) |
| | L63P, A71V, V82A | |
| NL4-3 296 | I50L + K45R, L63P, A71V, G73S, | 7.1 ± 1.6 (9.5) |
| | V77I, I93L | |
| NL4-3 020 | I50L + M46I, L63P, H69Q, V77I, | 1.9 ± 0.5 (2.5) |
| | I93L | |
| NL4-3 372 | I50L + L10I, L63P, G73S, I93L | 9.8 ± 0.7 (13) |
| NL4-3 450 | I50L + L19I, R41K, A71V, T74S | 4.4 ± 2.9 (5.9) |
| NL4-3 100A | I50L + L63P, K70R, A71V, N88S | 22.9 ± 6.4 (31) |
| NL4-3 100B | I50L + L63P, K70R, A71V, G73S | 27.8 ± 2.8 (37) |
| NL4-3 Control | | 1.5 ± 0.1 |
| NL4-3 | I50L + A71V | 8.6 ± 1.9 (5.9) |
| NL4-3 | A71V | 2.4 ± 0.04 (1.6) |
| NL4-3 | I50L | 3.1 ± 1.6 (2.1) |
| RF Control | | 0.7 ± 0.1 |
| RF | I50L + A71V | 4.0 ± 0.3 (5.7) |
| RF | A71V | 0.6 ± 0.2 (0.9) |
| RF | I50L | 2.2 ± 1.8 (3) |
| LAI Control | | 1.5 ± 0.3 |
| LAI | 150L + A71V | 15.1 ± 4.7 (10) |
| LAI | A71V | 3.7 ± 0.9 (2.4) |
| LAI | I50L | 8.2 ± 3.4 (5.4) |

TABLE 3
Progression of Isolates Following I50L Emergence

| Subject | | Susceptibility* | | | | | | | | RNA |
|---|---|---|---|---|---|---|---|---|---|---|
| ID | Week | ATV | APV | IDV | LPV | NFV | RTV | SQV | Substitutions | Load |
| 076 | 0.1 | | | | | | | | nd | |
| | 80 | 9.5 | 0.6 | 0.4 | 0.6 | 0.4 | 0.3 | 0.3 | 12S, 15V, 19I, | 4,630 |
| | | | | | | | | | 20R, 35D, | |
| | | | | | | | | | 36I, 41K, 50L, | |
| | | | | | | | | | 69K, 89M, | |
| | | | | | | | | | 93L | |
| | 92 | 16 | 0.9 | 0.5 | 0.5 | 0.6 | 0.3 | 0.5 | No Change | 5,240 |
| 169 | 0.1 | 1.1 | 0.6 | 0.8 | | 1.0 | 1.5 | 0.8 | 12S, 15V, 19I, | 59,400 |
| | | | | | | | | | 20R, 35D, | |
| | | | | | | | | | 36I, 41K, | |
| | | | | | | | | | 69K, 89M, | |
| | | | | | | | | | 93L | |
| | 90 | 12 | 0.7 | 0.5 | | 0.5 | 0.3 | 0.5 | +16E, +50L | 1,640 |
| | 102 | 9.2 | 0.6 | 0.4 | | 0.5 | 0.3 | 0.4 | +64V | 2,010 |
| | 114 | 13 | 1.2 | 0.4 | | 0.7 | 0.4 | 0.5 | +33F | 1,350 |
| | 0.1 | 1.3 | 1.0 | 1.3 | | 2.3 | 1.1 | 1.0 | 12S, 37H, | 229,575 |
| | | | | | | | | | 41K, 57K, | |
| | | | | | | | | | 63P, 77I, 93L | |
| 362 | 84** | | | | | | | | +45H/Q, +46I, | 1,987 |
| | | | | | | | | | +50L, +53L | |
| | | | | | | | | | +72T | |
| | 108 | 12 | 1.1 | 0.7 | | 1.1 | 0.4 | 0.5 | 45Q | 2,232 |
| | 120** | | | | | | | | No Change | 1,824 |
| 100 | 1.7 | 1.5 | 1.5 | 1.6 | | 2.1 | 2.2 | 1.3 | 63P, 70K/R | 3,432 |
| | 52** | | | | | | | | +50L, +71V, | 6,659 |
| | | | | | | | | | +73S, +88S | |
| | 104** | | | | | | | | +45R, +82A, | 5,803 |
| | | | | | | | | | +93L | |

nd = not determined
*Fold Change in EC₅₀ relative to reference strain
**Phenotypes lacking due to low (<1%) RC (single cycle replicative capacity)

TABLE 4
Phenotypic Impact of I50L and I50V on ATV and APV Susceptibility

| Isolates with | Susceptibility* | | Isolates with | Susceptibility* | |
|---|---|---|---|---|---|
| I50L | ATV | APV | I50V | ATV | APV |
| 1 | 3.5 | 0.3 | 1 | 0.3 | 3.5 |
| 2 | 4.9 | 0.6 | 2 | 0.4 | 4.2 |
| 3 | 5.2 | 0.5 | 3 | 0.6 | 8.0 |
| 4 | 5.8 | 0.4 | 4 | 1.2 | 9.4 |
| 5 | 6.7 | 0.6 | 5 | 0.4 | 11 |
| 6 | 7.9 | 0.4 | 6 | 0.8 | 11 |
| 7 | 8.6 | 0.6 | 7 | 0.5 | 12 |
| 8 | 8.8 | 0.8 | 8 | 0.5 | 14 |
| 9 | 9.5 | 0.6 | | | |
| 10 | 10 | 0.3 | | | |
| 11 | 11 | 0.4 | | | |
| 12 | 12 | 0.7 | | | |
| 13 | 13 | 0.7 | | | |
| 14 | 19 | 0.8 | | | |
| 15 | 22 | 0.9 | | | |
| 16 | 29 | 0.4 | | | |

*Fold Change in EC₅₀ relative to reference strain.

### REFERENCES

1. Hertogs K, Bloor S, Kemp S D, et al. Phenotypic and genotypic analysis of clinical HIV-1 isolates reveals extensive protease inhibitor cross-resistance: a survey of over 6000 samples. AIDS 2000; 14: 1203-1210
2. Voelker R. HIV drug resistance [news]. JAMA 2000; 284: 169
3. Martinez-Picado J, Savara A V, Shi L, Sutton L and D'Aquila R T. Fitness of human immunodeficiency virus type 1 protease inhibitor-selected single mutants. Virology 2000; 275: 318-322
4. Pieniazek D, Rayfield M, Hu D J, et al. Protease sequences from HIV-1 group M subtypes A-H reveal distinct amino acid mutation patterns associated with protease resistance in protease inhibitor-naive individuals worldwide. HIV Variant Working Group. AIDS 2000; 14: 1489-1495
5. Schmidt B, Walter H, Moschik B, et al. Simple algorithm derived from a geno-/phenotypic database to predict HIV-1 protease inhibitor resistance. AIDS 2000; 14: 1731-1738
6. Rabasseda X, Silvestre J and Castafier J. BMS-232632. Drugs of the Future 1999; 24: 375-380
7. Robinson B S, Riccardi K A, Gong Y F, et al. BMS-232632, a highly potent human immunodeficiency virus protease inhibitor that can be used in combination with other available antiretroviral agents. Antimicrob Agents Chemother 2000; 44: 2093-2099
8. Sanne I, Cahn P, Percival L, et al. Comparative results (Phase II 48-week): BMS-232632, Stavudine, Lamivudine as HAART for treatment-naive HIV(+) patients (AI424-008). 41st Interscience Conference on Antimicrobial Agents and Chemotherapy 2001
9. Squires K E, Thiry A and Giordano M. Atazanavir (ATV) QD and Efavirenz (EFV) QD with fixed-dose ZDV+3TC: Combination of antiviral efficacy and safety through week 24 (AI424-034). In: 42nd ICAAC Meeting. San Diego, Calif., 2002
10. Gong Y F, Robinson B S, Rose R E, et al. In vitro resistance profile of the human immunodeficiency virus type 1 protease inhibitor BMS-232632. Antimicrob Agents Chemother 2000; 44: 2319-2326
11. Colonno R J, Thiry A, Limoli K and Parkin N. Activities of Atazanavir (BMS-232632) against a Large Panel of Human Immunodeficiency Virus Type 1 Clinical Isolates Resistant to One or More Approved Protease Inhibitors. Antimicrob. Agents Chemother. 2003; 47: 1324-1333
12. Sanne I, Piliero P, Squires K, Thiry A, Schnittman S and Group A-CT. Results of a phase 2 clinical trial at 48 weeks (AI424-007): A dose-ranging, safety, and efficacy comparative trial of atazanavir at three doses in combination with didanosine and stavudine in antiretroviral-naive subjects. J AIDS 2003; 32: 18-29
13. Murphy R, al. e. Atazanavir, a once-daily protease inhibitor that does not elevate lipids and triglyceride concentrations: phase II 48-week safety and efficacy vs. nelfinavir, each in combination with stavudine and lamivudine in treatment-naive subjects (AI424-008). AIDS 2003; in press
14. Murphy R, Pokrovskiy V, Rozenbaum W, et al. Long-term efficacy and safety of atazanavir (ATV) with stavudine (d4T) and lamivudine (3TC) in patients previously treated with nelfinavir (NFV) or ATV: 108-week results of BMS study 008/044. In: 10th Conference on Retroviruses and Opportunistic Infections. Boston, Mass., 2003
15. Johnson V A, Byington R E. Infectivity assay (virus yield assay). New York: Stockton Press, 1990 (Walker B D, ed. Techniques in HIV Research)
16. Parkin N, Chappey C, Maroldo L, Bates M, Hellmann N and Petropoulos C. Phenotypic and genotypic HIV-1 drug resistance assays provide complementary information. J AIDS 2002; 31: 128-36
17. Petropoulos C J, Parkin N T, Limoli K L, et al. A Novel Phenotypic Drug Susceptibility Assay for Human Immunodeficiency Virus Type 1. Antimicrob. Agents Chemother. 2000; 44: 920-928
18. Potts B. Mini Reverse Transcriptase (RT) Assay. In: Walker B, ed. Techniques in HIV research. New York, N.Y.: Stockton Press, 1990: 103-106
19. Bally F, Martinez R, Peters S, Sudre P and Telenti A. Polymorphism of HIV type 1 gag p7/p1 and p1/p6 cleavage sites: clinical significance and implications for resistance to protease inhibitors. AIDS Res Hum Retroviruses 2000; 16: 1209-1213
20. Cote H C F, Brumme Z L and Harrigan P R. Human Immunodeficiency Virus Type 1 Protease Cleavage Site Mutations Associated with Protease Inhibitor Cross-Resistance Selected by Indinavir, Ritonavir, and/or Saquinavir. J. Virol. 2001; 75: 589-594
21. Doyon L, Croteau G, Thibeault D, Poulin F, Pilote L and Lamarre D. Second locus involved in human immunodeficiency virus type 1 resistance to protease inhibitors. J. Virol. 1996; 70: 3763-3769
22. Mammano F, Petit C and Clavel F. Resistance-Associated Loss of Viral Fitness in Human Immunodeficiency Virus Type 1: Phenotypic Analysis of Protease and gag Coevolution in Protease Inhibitor-Treated Patients. J. Virol. 1998; 72: 7632-7637.
23. Zhang Y, Imamichi H, Imamichi T, et al. Drug resistance during indinavir therapy is caused by mutations in the protease gene and in its Gag substrate cleavage sites. J. Virol. 1997; 71: 6662-6670
24. Watkins T, Resch W, Irlbeck D and Swanstrom R. Selection of High-Level Resistance to Human Immunodeficiency Virus Type 1 Protease Inhibitors. Antimicrob. Agents Chemother. 2003; 47: 759-769
25. Maguire M F, Guinea R, Griffin P, et al. Changes in Human Immunodeficiency Virus Type 1 Gag at Positions L449 and P453 Are Linked to I50V Protease Mutants In Vivo and Cause Reduction of Sensitivity to Amprenavir and Improved Viral Fitness In Vitro. J. Virol. 2002; 76: 7398-7406
26. Hirsch M S, Brun-Vezinet F, D'Aquila R T, et al. Antiretroviral drug resistance testing in adult HIV-1 infection: recommendations of an International AIDS Society-USA Panel. JAMA 2000; 283: 2417-2426
27. Patick A K, Duran M, Cao Y, et al. Genotypic and Phenotypic Characterization of Human Immunodeficiency Virus Type 1 Variants Isolated from Patients Treated with the Protease Inhibitor Nelfinavir. Antimicrob. Agents Chemother. 1998; 42: 2637-2644
28. Quinones-Mateu M, Arts E. Fitness of drug resistant HIV-1: methodology and clinical implications. Drug Resistance Updates 2002; 5: 224-233
29. Shenderovich M D, Kagan R M, Heseltine P N R and Ramnarayan K. Structure-based phenotyping predicts HIV-1 protease inhibitor resistance. Protein Sci 2003; 12: 1706-1718
30. Maguire M, Shortino D, Klein A, et al. Emergence of Resistance to Protease Inhibitor Amprenavir in Human Immunodeficiency Virus Type 1-Infected Patients: Selection of Four Alternative Viral Protease Genotypes and Influence of Viral Susceptibility to Coadministered Reverse Transcriptase Nucleoside Inhibitors. Antimicrob. Agents Chemother. 2002; 46: 731-738
31. Tisdale M, Myers R, Maschera B, Parry N, Oliver N and Blair E. Cross-resistance analysis of human immunodeficiency virus type 1 variants individually selected for resistance to five different protease inhibitors. Antimicrob. Agents Chemother. 1995; 39: 1704-1710
32. Resch W, Ziermann R, Parkin N, Gamarnik A and Swanstrom R. Nelfinavir-Resistant, Amprenavir-Hypersusceptible Strains of Human Immunodeficiency Virus Type 1 Carrying an N88S Mutation in Protease Have Reduced Infectivity, Reduced Replication Capacity, and Reduced Fitness and Process the Gag Polyprotein Precursor Aberrantly. J. Virol. 2002; 76: 8659-8666
33. Colonno, R. et al, “Identification of Amino Acid Substitutions Correlated with Reduced Atazanavir Susceptibility in Patients Treated with Atazanavir Containing Regimens,” 6^{th} International Conference Workshop on HIV Drug Resistance, Jul. 2-6, 2002, Seville, Spain (Abstract only).

### List of Abbreviations

3TC lamivudine
A alanine
ABC abacavir
APV amprenavir
ATV atazanavir
ATV/r atazanavir boosted with ritonavir
AZT or ZDV zidovudine
D aspartic acid
DDI didanosine
D4T stavudine
EC₅₀ concentration of drug required to reduce viral replication 50%
EFV efavirenz
F phenylalanine
FC fold change in susceptibility compared to reference strain
G glycine
HAART highly active anti-retroviral therapy
HIV human immunodeficiency virus
I or Ile isoleucine
IDV indinavir
L or Leu leucine
LPV lopinavir
LPV/r lopinavir boosted with ritonavir
MOI multiplicity of infection (measurement of the amount of virus used)
N asparagine
NNRTI non-nucleoside reverse transcriptase inhibitor
NFV nelfinavir
P proline
PI protease inhibitor
R arginine
RTV ritonavir
S serine
SQV saquinavir
V or val valine
Y tyrosine

ATV has a distinct resistance profile relative to other marketed PIs. An I50L substitution appears to be the key signature resistance substitution for ATV and emerged in all isolates obtained from treatment-naïve patients and in 30% of isolates from treatment-experienced patients who develop ATV resistance while on treatment regimens containing ATV as the primary PI. In contrast, phenotypic and genotypic evaluation of viral isolates from patients receiving ATV/SQV-containing regimens showed no evidence of the I50L substitution. In these patients, isolates displayed decreased susceptibility to multiple PIs along with resistance to both ATV and SQV. This was an expected result, since presence of the I50L substitution would result in resistance to ATV, but have increased susceptibility to SQV. A similar pathway was also observed with 70% of ATV or ATV/r treated patients who developed resistance in the absence of an I50L substitution. Clinical isolates containing the I50L substitution developed resistance specifically to ATV, while increasing phenotypic susceptibility to the currently approved PIs. A phenotypic and genotypic analysis of baseline isolates becoming resistant to ATV via the I50L or alternative pathways failed to identify any characteristic, which strongly predicted one pathway or another. In general, isolates showing greater susceptibility to ATV and possessing fewer PI resistant substitutions tended to become resistant to ATV by introducing an I50L change. The finding of enhanced susceptibility to other PIs when an I50L substitution is present is quite unique. Future PI treatment options for HIV infected patients that develop an I50L mutation are now offered as disclosed by the invention herein claimed.

The term “pharmaceutically acceptable salt” as used herein and in the claims is intended to include nontoxic base addition salts. Suitable salts include those derived from organic and inorganic acids such as, without limitation, hydrochloric acid, hydrobromic acid, phosphoric acid, sulfuric acid, methanesulfonic acid, acetic acid, tartaric acid, lactic acid, sulfinic acid, citric acid, maleic acid, fumaric acid, sorbic acid, aconitic acid, salicylic acid, phthalic acid, and the like. The term “pharmaceutically acceptable salt” as used herein is also intended to include salts of acidic groups, such as a carboxylate, with such counterions as ammonium, alkali metal salts, particularly sodium or potassium, alkaline earth metal salts, particularly calcium or magnesium, and salts with suitable organic bases such as lower alkylamines (methylamine, ethylamine, cyclohexylamine, and the like) or with substituted lower alkylamines (e.g. hydroxyl-substituted alkylamines such as diethanolamine, triethanolamine or tris(hydroxymethyl)-aminomethane), or with bases such as piperidine or morpholine.

In the method of the present invention, the term “therapeutically or antiviral effective amount” means the total amount of each active component of the method that is sufficient to show a meaningful patient benefit, i.e., healing of acute conditions characterized by inhibition of the HIV infection. When applied to an individual active ingredient, administered alone, the term refers to that ingredient alone. When applied to a combination, the term refers to combined amounts of the active ingredients that result in the therapeutic effect, whether administered in combination, serially or simultaneously. The terms “treat, treating, treatment” as used herein and in the claims means preventing or ameliorating diseases associated with HIV infection.

With regard to atazanavir and the other approved and already marketed protease inhibitors-saquinavir, ritonavir, indinavir, nelfinavir, amprenavir, fosamprenavir, lopinavir and Kaletra® (combination of ritonavir and lopinavir)-it is contemplated that these drugs would be used in accordance with the claimed invention herein in the usually approved dosage levels and methods of administration and form as is commercially available.

As for other protease inhibitors, the following would apply.

In the method of the present invention the protease inhibitors may be administered orally, parenterally (including subcutaneous injections, intravenous, intramuscular, intrasternal injection or infusion techniques), by inhalation spray, or rectally, in dosage unit formulations containing conventional non-toxic pharmaceutically acceptable carriers, adjuvants and diluents.

Thus, in accordance with the present invention, there is further provided a method of treating HIV infection and AIDS. The treatment involves administering to a patient in need of such treatment therapeutically effective amounts of atazanavir and protease inhibitor.

The protease inhibitors are usually in the form of a pharmaceutical composition which may be in the form of orally administrable suspensions or tablets; nasal sprays, sterile injectable preparations, for example, as sterile injectable aqueous or oleagenous suspensions or suppositories.

When administered orally as a suspension, these compositions are prepared according to techniques well known in the art of pharmaceutical formulation and may contain microcrystalline cellulose for imparting bulk, alginic acid or sodium alginate as a suspending agent, methylcellulose as a viscosity enhancer, and sweetners/flavoring agents known in the art. As immediate release tablets, these compositions may contain microcrystalline cellulose, dicalcium phosphate, starch, magnesium stearate and lactose and/or other excipients, binders, extenders, disintegrants, diluents, and lubricants known in the art.

The injectable solutions or suspensions may be formulated according to known art, using suitable non-toxic, parenterally acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

The protease inhibitors used in accordance with the methods of this invention can be administered orally to humans in a dosage range of 1 to 100 mg/kg body weight in divided doses. One preferred dosage range is 1 to 10 mg/kg body weight orally in divided doses. Another preferred dosage range is 1 to 20 mg/kg body weight in divided doses. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

1. A method of treating HIV infection in a human patient wherein the infecting HIV strain has become resistant to atazanavir, the method comprising administration of a therapeutically effective amount of atazanavir or a pharmaceutically acceptable salt thereof, and a therapeutically effective amount of at least one other HIV protease inhibitor.

2. The method of claim 1 wherein said resistance is manifested by the existence of a signature mutation consisting of the I50L mutation in the HIV protease and the amount of atazanavir or a pharmaceutically acceptable salt thereof is sufficient to maintain the existence of the I50L mutation in the HIV protease.

3. The method of claim 1 wherein the other HIV protease inhibitor is selected from the group consisting of saquinavir, ritonavir, indinavir, nelfinavir, amprenavir, fosamprenavir and lopinavir.

4. The method of claim 1 wherein the amount of the other HIV protease inhibitor administered to the human patient in combination with the atazanavir or pharmaceutically acceptable salt thereof is less than the amount required in the absence of atazanavir.

5. The method of claim 4 wherein the other HIV protease inhibitor is selected from the group consisting of saquinavir, ritonavir, indinavir, nelfinavir, fosamprenavir, amprenavir and lopinavir.

6. A method for enhancing the effectiveness of a second HIV protease inhibitor in treating HIV infection in a human patient whose HIV strain has become resistant to atazanavir or a pharmaceutically acceptable salt thereof, comprising administering to said human patient an amount of atazanavir or a pharmaceutically acceptable salt thereof effective in maintaining the resistant strain, in combination with a therapeutically effective amount of the second HIV protease inhibitor.

7. The method of claim 6 wherein said resistance is manifested by the existence of a signature mutation consisting of the I50L mutation in the HIV protease and the amount of atazanavir or a pharmaceutically acceptable salt thereof is sufficient to maintain the existence of the I50L mutation in the HIV protease.

8. The method of claim 6 wherein said HIV protease inhibitor is selected from the group consisting of saquinavir, ritonavir, indinavir, nelfinavir, fosamprenavir, amprenavir and lopinavir.

9. The method of claim 6 wherein the amount of the other HIV protease inhibitor administered to the human patient in combination with the atazanavir or pharmaceutically acceptable salt thereof is less than the amount required in the absence of atazanavir.

10. The method of claim 9 wherein the other HIV protease inhibitor is selected from the group consisting of saquinavir, ritonavir, indinavir, nelfinavir, fosamprenavir, amprenavir and lopinavir.
